(19) United States
(12) Patent Application Publication
(10) Pub. No.: US 2018/0064349 A1
(43) Pub. Date: Mar. 8, 2018

(54) PORTABLE WIRELESS THERMOMETER WITH LOCATION DETERMINATION

(71) Applicant: Prolego Health, Inc., Ivoryton, CT (US)

(72) Inventor: Walter Kerr, Berkeley, CA (US)

(21) Appl. No.: 15/693,408

(22) Filed: Aug. 31, 2017

Related U.S. Application Data
(60) Provisional application No. 62/384,080, filed on Sep. 6, 2016.

(51) Int. Cl. A61B 5/01; H04B 1/3827; G01J 5/00; A61B 5/00

(52) U.S. Cl. CPC A61B 5/015; H04B 1/3833; G01J 5/0025; A61B 5/0075; G01S 19/42; A61B 5/0008; A61B 5/0013; A61B 5/0022; A61B 5/0077

(57) ABSTRACT

A handheld measurement device includes a temperature detector for measuring infrared energy of a mammal; a location determination mechanism for determining a location of said device when in use; memory storing measured temperature and corresponding location information; and a communication mechanism constructed and adapted to send multiple stored measured temperature and corresponding location data to a remote location.

### RELATED APPLICATIONS

This application is related to and claims priority from U.S. Provisional Patent Application No. 62/384,080, titled “Portable Wireless Thermometer With Location Determination,” filed Sep. 6, 2017, the entire contents of which are fully incorporated herein by reference for all purposes.

### COPYRIGHT STATEMENT

This patent document contains material subject to copyright protection. The copyright owner has no objection to the reproduction of this patent document or any related materials in the files of the United States Patent and Trademark Office, but otherwise reserves all copyrights whatsoever.

### FIELD OF THE INVENTION

This invention relates to the collection and analysis of medical data, and, more specifically, to devices, systems, and methods for the collection and analysis of data in a region in order to evaluate whether the data are indicative of a potential health issue in that region.

### BACKGROUND

It is desirable, especially in remote areas, to detect infectious disease outbreaks at a community level, before they spread to other regions or communities. Such detection may be used by public health officials and the like to prevent the spread of infectious diseases.

Fever is a common sign of illnesses in mammals, and febrile data is useful in determining and evaluating illnesses. A high fever, relative to some norm, may be indicative of an illness in an individual. High fevers in multiple individuals in a group, relative to a norm, may be indicative of a potential health issue (e.g., a potential infectious disease outbreak) within that group.

In remote or rural areas it is often difficult to obtain useful febrile or other medical data. The data typically need to be collected by non-technical people and are often subject to error and local interpretation.

It is desirable and an object of this invention to provide a mechanism that allows reliable and easy collection of febrile or other medical data from remote communities. It is further desirable, and a further object of this invention, to provide mechanisms, systems, and methods to analyze collected data in order to evaluate whether the data are indicative of a potential health issue.

### BRIEF DESCRIPTION OF THE DRAWINGS

Other objects, features, and characteristics of the present invention as well as the methods of operation and functions of the related elements of structure, and the combination of parts and economies of manufacture, will become more apparent upon consideration of the following description and the appended claims with reference to the accompanying drawings, all of which form a part of this specification.

FIG. 1 depicts aspects of a portable wireless thermometer according to exemplary embodiments hereof;

FIG. 2 shows an exemplary data structure according to exemplary embodiments hereof;

FIG. 3 depicts aspects of operation of system according to exemplary embodiments hereof; and

FIG. 4 depicts aspects of computing according to exemplary embodiments hereof.

### DETAILED DESCRIPTION OF THE PRESENTLY PREFERRED EXEMPLARY EMBODIMENTS

### Glossary

As used herein, unless used otherwise, the following terms or abbreviations have the following meanings:

GPS means global positioning system.

A “mechanism” refers to any device(s), process(es), routine(s), service(s), module(s), or combination thereof. A mechanism may be implemented in hardware, software, firmware, using a special-purpose device, or any combination thereof. A mechanism may be integrated into a single device or it may be distributed over multiple devices. The various components of a mechanism may be co-located or distributed. The mechanism may be formed from other mechanisms. In general, as used herein, the term “mechanism” may thus be considered shorthand for the term device(s) and/or process(es) and/or service(s).

### Description

With reference now to FIG. 1, a measurement device 100 according to exemplary embodiments hereof includes one or more sensors 102, communications mechanisms 104, one or more processors 106, memory 108, and one or more location mechanisms 110. The device is preferably handheld.

The sensors 102 include one or more temperature sensors 112 which may be used to obtain non-contact and/or contact temperature measurements of a subject. In presently preferred implementations, the temperature sensor(s) 112 include at least one non-contact temperature measurement sensor (e.g., via infrared sensor, a thermal sensor, or the like). It should be appreciated that, depending on what a practitioner wants to measure, a thermal sensor can yield more accurate results than an infrared sensor. In some embodiments the temperature sensors 112 may include a sensor to measure an ambient temperature of the device.

The sensors may also include a camera 114 or the like to obtain image data if required (and permitted). It should be appreciated that the collection of medical data in some areas may be subject to local concerns and taboos, and the collection of image data may not be allowed.

The communications mechanisms 104 preferably include mechanisms 116 that support remote (e.g., wireless or radio) communication and wired communication 118. The wireless/radio communication mechanisms 116 may include one or more of satellite 120, cellular 122, Bluetooth 124, and WiFi 126 mechanisms. In preferred implementations the communication mechanisms 116 include at least satellite mechanisms 120. The wire communication mechanism(s) may include USB communication and/or Ethernet network mechanisms, or the like.

The location mechanism 110 preferably includes a GPS mechanism. As is well known, GPS is a radio navigation system that allows land, sea, and airborne users to determine their exact location and time, in all weather conditions, anywhere in the world. If necessary, multiple GPS mechanisms may be included in order to obtain accurate location and time measurements, regardless of which underlying GPS system is used. In order to conserve power in the device 100, the GPS mechanism(s) may be turned off when the device is not in use or taking sensor (temperature) measurements. The GPS mechanism(s) 110 may be used to provide accurate time and date information to the device and may be used to synchronize a device clock (not shown).

The device 100 may measure temperature passively and/or actively. In a passive mode, the device 100 collects temperature data via temperature sensor 112 (e.g., an infrared sensor) if someone passes in front of the device. In an active mode, a user pushes a button (not shown) or the like to initiate a temperature measurement.

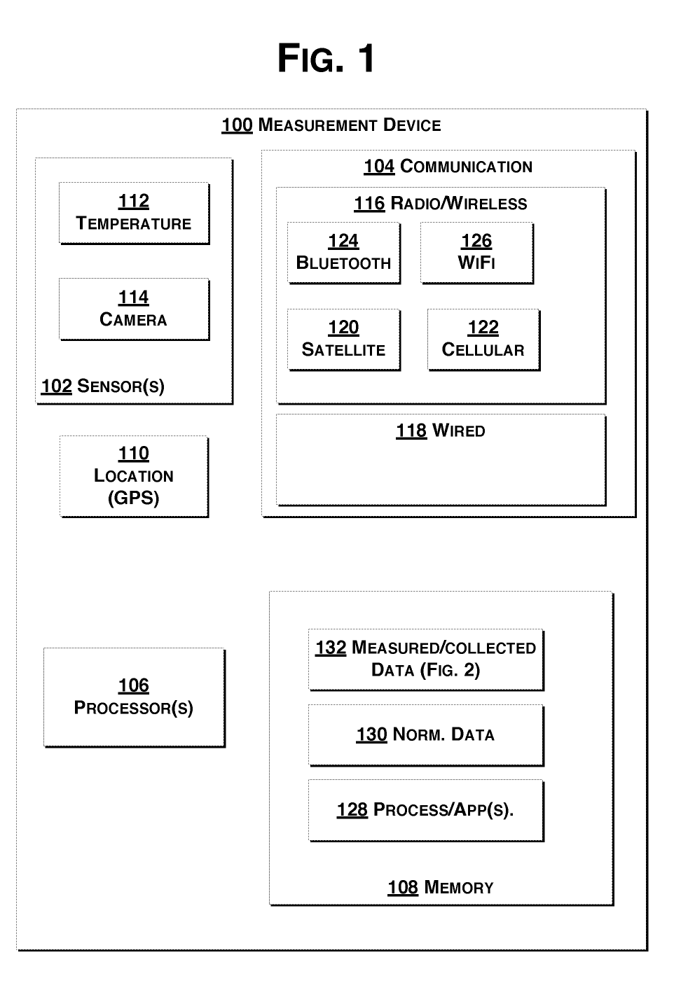

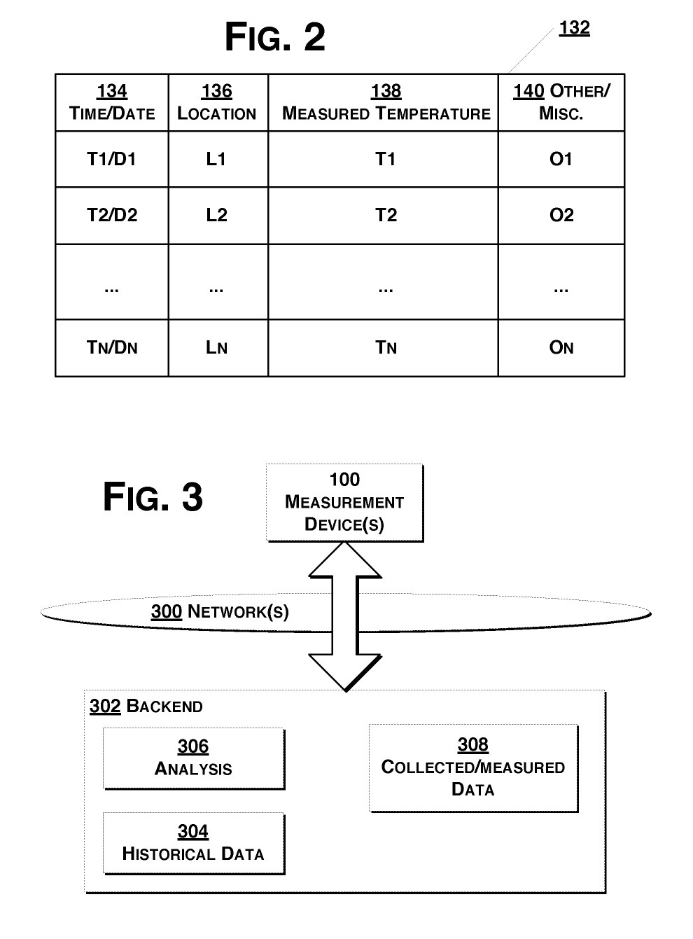

The memory 108 may store software applications 128 that support operation and use of the device. These applications 128 may include user interface applications that support collection of measurement data and transmission (or otherwise providing) collected measurement data to a remote location (as described below). The memory 108 may also contain information 130 about normal data for the region(s) in which the device is being used. This normal data 130 may be used to perform localized (in device) checking of measured data. The memory 108 may store measured/collected data 132, e.g. as shown in the exemplary data structure in FIG. 2.

In addition to temperature data, in some embodiments, the device 100 may collect and transmit other information, (for example, diarrhea rates, jaundice rates, etc.).

As shown in FIG. 2, the measured/collected data 132 may include, for each measurement, a time and date 134 (e.g., obtained from a device clock or from the GPS mechanism 110), location information 136 (also obtained from the GPS mechanism 110), a measured temperature data 138, and, optionally, other/miscellaneous information 140. The other/miscellaneous information 140 may include annotations or comments made by the user (e.g., via a user interface of the device) and/or other sensor information (e.g., ambient temperature data or the like).

In some embodiments the device may be used in conjunction with a second device (e.g., a cell phone, satellite phone, GPS device, or the like) and may obtain its location information from that second device. For example, the device 100 may be connected (by wire or wirelessly) to a co-located device that includes a location mechanism such as a GPS. The device 100 may query location information from the second device.

The device 100 is preferably battery-powered, allowing a user (e.g., a health practitioner) to take it into the field, including in areas with limited or no electricity. In areas with more reliable power sources, the device can also be plugged in for use with local or external power supply.

The device 100 may include a display and input mechanism (not shown) to provide a user interface and support control and operation of the device.

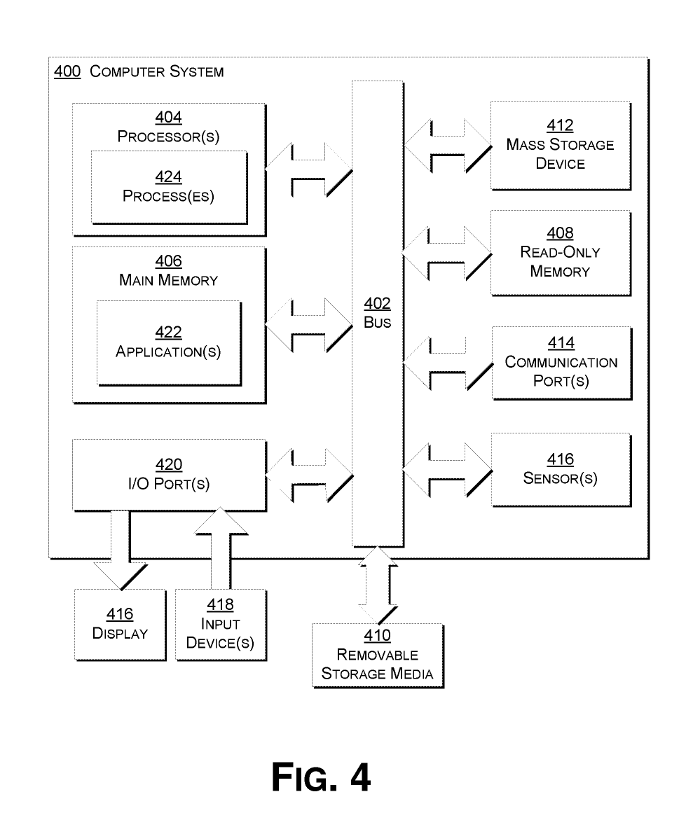

With reference now to FIG. 3, measurement device(s) 100 communicate (e.g., via one or more networks 300) with a backend 302. Communication with the backend 302 uses the communication mechanism(s) in the device and may be done in real time or on demand. The network(s) 300 may include satellite networks, cellular networks, etc., and the device connects to the backend 302 in a suitable manner given its location. In order to simplify operation and use of the device 100, preferably a device 100 communicates with the backend 302 automatically, without operator intervention.

The backend 302 includes historical data 304 stored in one or more databases, and one or more analysis mechanisms 306. The historical data 304 are preferably stored or accessible by location. Newly measured data (obtained from one or more devices 100) may be stored in one or more databases 308 on the backend. The system is not limited by the manner in which the databases are formed or implemented. For example, the database(s) 308 may be in any form, may be distributed, and may use any database interface and software. The analysis mechanism(s) 306 may use or be integrated with existing analysis software or the like. The system is not limited by the manner in which any analysis mechanism 306 is implemented or integrated with the database(s) 308.

When a device 100 connects with the backend (e.g., via network(s) 300), the device provides its measured data 132 to the backend that stores those data as newly measured data 308. The analysis mechanisms 306 then analyze and compare the newly measured data 308 to the historical data 304. Preferably the newly measured data 308 is compared to historical data 304 for the same location (e.g., region).

The newly measured data 308 may be integrated into the historical data 304, preferably after verification and integrity checking.

In a presently preferred implementation, the device 100 includes a satellite module 120 that communicates with low-orbit satellites connected to the Iridium satellite network, allowing a user/practitioner to take the device to areas with little to no Internet connectivity. Through this satellite connection, information may be sent to/from the device 100, including temperature data, GPS coordinates, and other health data collected in the field. Depending on the communications infrastructure where the device is being used, a device's satellite communications module can be replaced with either a WiFi module, Ethernet module, or SMS-module for communicating data to/from the device.

In some embodiments a second device (e.g., a cellular phone, satellite phone, or the like) may be used to provide the remote/wireless functionality of the device 100. For example, the device 100 may be connected (by wire or wirelessly) to a co-located second device that includes remote connection functionality (e.g., a cellular or satellite phone), and the second device may be used for remote connection to the backend 302.

### Discussion & Operation

As described, in preferred embodiments, the device is a handheld thermometer with an infrared and/or thermal sensor to capture a human or animal's temperature. Because the device uses a non-contact based approach to capture temperature, a health practitioner can use the device to conduct mass surveillance in a short period, while limiting potential the spread of harmful microorganisms from one subject to another.

In an exemplary operation, each time a user (e.g., a health practitioner) clicks a button on the device to capture a subject's temperature, the temperature is displayed on the device for the health practitioner to see and a record is created and saved to the device's memory 108 as measured data 132. The record preferably includes: a) the time and date the measurement (e.g., temperature) was recorded; b) the subject's temperature; and c) the location (e.g., GPS coordinates) where the reading was taken.

In some embodiments, the device may be customized to meet health practitioners' specific public health surveillance needs. In these embodiments, the device may include input mechanisms (e.g., a keyboard or additional buttons) for the health practitioner to use to answer prompts displayed on the device. For example, in the case of a health practitioner surveying new mothers about their newborns' health, in addition to capturing temperature data, a series of questions can be loaded on to the device for the practitioner to answer about the subject. (Example questions/answers may be: “Does the subject have diarrhea? Yes/No.”; “Does the subject have jaundice? Yes/No.”) The buttons may be programmed to allow a practitioner to choose from one of a series of options. These prompts can be represented for the local health practitioner in either text or graphic form to be intuitive and easy-to-use, including for those health practitioners who may have limited reading or technical skills. For example, for health practitioners collecting animal febrile data, the user may select the species that was surveyed from a series of options presented in either text or graphic form. These prompt responses are saved to memory (as other/misc. data 140) along with the temperature, GPS, and time/date information.

Cloud-Based Software

The device 100 may send its data to cloud-based software (e.g., analysis mechanisms 306 on backend 302) that can interpret data, present it in a readable, user-friendly format, and identify and predict potential infectious disease outbreaks from the data.

The device 100 preferably comes pre-loaded with information (e.g., as norm data 130) about expected distributions of fevers among healthy human or animal populations in communities where the device will be used. For example, in areas with high rates of malaria, there will be a higher distribution of people with fevers, and that number of people with fevers in a community may fluctuate with wet or dry seasons. If, after a statistically significant amount of data has been collected from a community, there appears to be a deviation from the expected distribution of temperature data, the software may alert appropriate public health officials about that fact. The application(s) 128 on the device 100 can thereby show that there is a higher-than-expected number of people in a given community with a fever (or other symptoms, if the device is used to report on other symptoms), and alert public health officials. The application(s) 128 thus provide an early warning system about potential infectious disease outbreak (e.g., a spike in fevers is an early indicator of infectious disease outbreak).

Because the device can collect and transmit other information, in addition to temperature data (for example, diarrhea rates, jaundice rates, etc.), public health officials can also use the software to view those data points. This vastly improves upon current data collection and analysis methods in developing areas, which are often paper-based or communicated via telephone, increasing the likelihood for human error and often inhibiting officials and scientists ability to view data in the aggregate to extrapolate trends.

### Computing

The services, mechanisms, operations and acts shown and described above are implemented, at least in part, by software running on one or more computers.

Programs that implement such methods (as well as other types of data) may be stored and transmitted using a variety of media (e.g., computer readable media) in a number of manners. Hard-wired circuitry or custom hardware may be used in place of, or in combination with, some or all of the software instructions that can implement the processes of various embodiments. Thus, various combinations of hardware and software may be used instead of software only.

One of ordinary skill in the art will readily appreciate and understand, upon reading this description, that the various processes described herein may be implemented by, e.g., appropriately programmed general purpose computers, special purpose computers and computing devices. One or more such computers or computing devices may be referred to as a computer system.

FIG. 4 is a schematic diagram of a computer system 400 upon which embodiments of the present disclosure (including the analysis mechanisms 306 of the backend 302) may be implemented and carried out.

According to the present example, the computer system 400 includes a bus 402 (i.e., interconnect), one or more processors 404, a main memory 406, read-only memory 408, removable storage media 410, mass storage 412, and one or more communications ports 414. Communication port 414 may be connected to one or more networks by way of which the computer system 400 may receive and/or transmit data.

As used herein, a “processor” means one or more microprocessors, central processing units (CPUs), computing devices, microcontrollers, digital signal processors, or like devices or any combination thereof, regardless of their architecture. An apparatus that performs a process can include, e.g., a processor and those devices such as input devices and output devices that are appropriate to perform the process.

Processor(s) 404 can be any known processor, such as, but not limited to, an Intel® Itanium® or Itanium 2® processor(s), AMD® Opteron® or Athlon MP® processor(s), or Motorola® lines of processors, and the like. Communications port(s) 414 can be any of an RS-232 port for use with a modem based dial-up connection, a 10/100 Ethernet port, a Gigabit port using copper or fiber, or a USB port, and the like. Communications port(s) 414 may be chosen depending on a network such as a Local Area Network (LAN), a Wide Area Network (WAN), a CDN, or any network to which the computer system 400 connects. The computer system 400 may be in communication with peripheral devices (e.g., display screen 416, input device(s) 418) via Input/Output (I/O) port 420.

Main memory 406 can be Random Access Memory (RAM), or any other dynamic storage device(s) commonly known in the art. Read-only memory 408 can be any static storage device(s) such as Programmable Read-Only Memory (PROM) chips for storing static information such as instructions for processor 404. Mass storage 412 can be used to store information and instructions. For example, hard disks such as the Adaptec® family of Small Computer Serial Interface (SCSI) drives, an optical disc, an array of disks such as Redundant Array of Independent Disks (RAID), such as the Adaptec® family of RAID drives, or any other mass storage devices may be used.

Bus 402 communicatively couples processor(s) 404 with the other memory, storage, and communications blocks. Bus 402 can be a PCI/PCI-X, SCSI, a Universal Serial Bus (USB) based system bus (or other) depending on the storage devices used, and the like. Removable storage media 410 can be any kind of external hard-drives, floppy drives, IOMEGA® Zip Drives, Compact Disc—Read Only Memory (CD-ROM), Compact Disc—Re-Writable (CD-RW), Digital Versatile Disk—Read Only Memory (DVD-ROM), etc.

Embodiments herein may be provided as one or more computer program products, which may include a machine-readable medium having stored thereon instructions, which may be used to program a computer (or other electronic devices) to perform a process. As used herein, the term “machine-readable medium” refers to any medium, a plurality of the same, or a combination of different media, which participate in providing data (e.g., instructions, data structures) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random access memory, which typically constitutes the main memory of the computer. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications.

The machine-readable medium may include, but is not limited to, floppy diskettes, optical discs, CD-ROMs, magneto-optical disks, ROMs, RAMs, erasable programmable read-only memories (EPROMs), electrically erasable programmable read-only memories (EEPROMs), magnetic or optical cards, flash memory, or other type of media/machine-readable medium suitable for storing electronic instructions. Moreover, embodiments herein may also be downloaded as a computer program product, wherein the program may be transferred from a remote computer to a requesting computer by way of data signals embodied in a carrier wave or other propagation medium via a communication link (e.g., modem or network connection).

Various forms of computer readable media may be involved in carrying data (e.g. sequences of instructions) to a processor. For example, data may be (i) delivered from RAM to a processor; (ii) carried over a wireless transmission medium; (iii) formatted and/or transmitted according to numerous formats, standards or protocols; and/or (iv) encrypted in any of a variety of ways well known in the art.

A computer-readable medium can store (in any appropriate format) those program elements that are appropriate to perform the methods.

As shown, main memory 406 is encoded with application(s) 422 that supports the functionality discussed herein (the application 422 may be an application that provides some or all of the functionality of the CD services described herein, including the client application and the optimization support mechanism 112). Application(s) 422 (and/or other resources as described herein) can be embodied as software code such as data and/or logic instructions (e.g., code stored in the memory or on another computer readable medium such as a disk) that supports processing functionality according to different embodiments described herein.

During operation of one embodiment, processor(s) 404 accesses main memory 406 via the use of bus 402 in order to launch, run, execute, interpret or otherwise perform the logic instructions of the application(s) 422. Execution of application(s) 422 produces processing functionality of the service related to the application(s). In other words, the process(es) 424 represent one or more portions of the application(s) 422 performing within or upon the processor(s) 404 in the computer system 400.

It should be noted that, in addition to the process(es) 424 that carries (carry) out operations as discussed herein, other embodiments herein include the application 422 itself (i.e., the un-executed or non-performing logic instructions and/or data). The application 422 may be stored on a computer readable medium (e.g., a repository) such as a disk or in an optical medium. According to other embodiments, the application 422 can also be stored in a memory type system such as in firmware, read only memory (ROM), or, as in this example, as executable code within the main memory 406 (e.g., within Random Access Memory or RAM). For example, application 422 may also be stored in removable storage media 410, read-only memory 408 and/or mass storage device 412.

Those skilled in the art will understand that the computer system 400 can include other processes and/or software and hardware components, such as an operating system that controls allocation and use of hardware resources.

As discussed herein, embodiments of the present invention include various steps or operations. A variety of these steps may be performed by hardware components or may be embodied in machine-executable instructions, which may be used to cause a general-purpose or special-purpose processor programmed with the instructions to perform the operations. Alternatively, the steps may be performed by a combination of hardware, software, and/or firmware. The term “module” refers to a self-contained functional component, which can include hardware, software, firmware or any combination thereof.

One of ordinary skill in the art will readily appreciate and understand, upon reading this description, that embodiments of an apparatus may include a computer/computing device operable to perform some (but not necessarily all) of the described process.

Embodiments of a computer-readable medium storing a program or data structure include a computer-readable medium storing a program that, when executed, can cause a processor to perform some (but not necessarily all) of the described process.

Where a process is described herein, those of ordinary skill in the art will appreciate that the process may operate without any user intervention. In another embodiment, the process includes some human intervention (e.g., a step is performed by or with the assistance of a human).

As used herein, including in the claims, the phrase “at least some” means “one or more,” and includes the case of only one. Thus, e.g., the phrase “at least some services” means “one or more services”, and includes the case of one service.

As used herein, including in the claims, the phrase “based on” means “based in part on” or “based, at least in part, on,” and is not exclusive. Thus, e.g., the phrase “based on factor X” means “based in part on factor X” or “based, at least in part, on factor X.” Unless specifically stated by use of the word “only”, the phrase “based on X” does not mean “based only on X.”

As used herein, including in the claims, the phrase “using” means “using at least,” and is not exclusive. Thus, e.g., the phrase “using X” means “using at least X.” Unless specifically stated by use of the word “only”, the phrase “using X” does not mean “using only X.”

In general, as used herein, including in the claims, unless the word “only” is specifically used in a phrase, it should not be read into that phrase.

As used herein, including in the claims, a list may include only one item, and, unless otherwise stated, a list of multiple items need not be ordered in any particular manner A list may include duplicate items. For example, as used herein, the phrase “a list of widgets” may include one or more widgets.

It should be appreciated that the words “first” and “second” in the description and claims are used to distinguish or identify, and not to show a serial or numerical limitation. Similarly, the use of letter or numerical labels (such as “(a)”, “(b)”, and the like) are used to help distinguish and/or identify, and not to show any serial or numerical limitation or ordering.

While the invention has been described in connection with what is presently considered to be the most practical and preferred embodiments, it is to be understood that the invention is not to be limited to the disclosed embodiment, but on the contrary, is intended to cover various modifications and equivalent arrangements included within the spirit and scope of the appended claims.

I claim:

1. A handheld measurement device comprising:
a temperature detector for measuring infrared energy of a mammal;
a location determination mechanism for determining a location of said device when in use;
memory storing measured temperature and corresponding location information;
a communication mechanism constructed and adapted to send multiple stored measured temperature and corresponding location data to a remote location.
